(19) United States
(12) Patent Application Publication
(10) Pub. No.: US 2007/0086959 A1
(43) Pub. Date: Apr. 19, 2007

(54) Use of a compositionally gradient copolymer in an aerosol device comprising two compartments, and aerosol device comprising said copolymer and a compressed gas

(75) Inventors: Jonathan Gawtrey, Boulogne (FR); Nathalie Mougin, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL, Paris (FR)

(21) Appl. No.: 10/568,882

(22) PCT Filed: Aug. 20, 2004
(86) PCT No.: PCT/FR04/02175

§ 371 Date: Oct. 31, 2006

(30) Foreign Application Priority Data
Aug. 22, 2003 (FR) 0310109

(51) Int. Cl. A61K 8/31

(52) U.S. Cl. 424/47; 128/200.22

(57) ABSTRACT

The invention relates to an aerosol device with two compartments, which contains the following: (a) in a first compartment, a hair treatment composition comprising, in a cosmetically-acceptable aqueous medium, at least one compositionally-graded copolymer comprising at least two different monomers and having a mass polydispersity index (Ip) of less than or equal to 2.5; and (b) in a second compartment, a compressed gas which is selected from air, nitrogen, carbon dioxide and mixtures thereof and, optionally, at least one liquefied gas. The sprayed product can be used, for example, to shape and/or hold styled hair.

The present invention relates to a two-compartment aerosol device comprising a specific hair treatment composition in one compartment and a compressed gas in the other compartment. It also relates to a use of a compositionally gradient copolymer in two-compartment aerosol devices comprising a compressed gas as propellant.

The most widespread hair products for the shaping and/or the form retention of the hairstyle in the cosmetics market are spray compositions essentially composed of a solution, generally an alcoholic solution, and of one or more materials, generally polymeric resins, referred to as fixing materials, the role of which is to form joins between the hairs, as a mixture with various cosmetic adjuvants. The fixing materials are generally fixing polymers, that is to say film-forming polymers which are soluble or dispersible in water or the alcohol, such as vinyl acetate/crotonic acid copolymers, anionic or amphoteric acrylic resins, polyurethanes, and the like.

For essentially ecological reasons, a search is underway to reduce the amount of volatile organic compounds (or VOCs) present in the composition. To reduce the amount of VOC and to obtain a low-VOC aerosol device, organic solvents, such as ethanol and dimethyl ether, are respectively partially replaced by water and a compressed gas.

However, there are disadvantages to the replacement of ethanol by water and dimethyl ether by a compressed gas, such as air, for example a deterioration in the quality of the spray, phenomena of blockage of the aerosol device and, sometimes, loss of the cosmetic performance.

The use of specific polymers, such as compositionally gradient copolymers, in an aqueous medium makes it possible to avoid these disadvantages but the formulation of these polymers alone with dimethyl ether or another liquefied gas as propellant results in bleaching of the hair.

The Applicant Company has found, surprisingly, that the use of a specific two-compartment aerosol device, the propellant of which is a specific compressed gas, optionally in combination with at least one liquefied gas, makes it possible to avoid bleaching of the hair and to obtain hair products for the shaping and/or the form retention of the hairstyle with a low VOC content.

A subject-matter of the present invention is thus a two-compartment aerosol device which comprises, in a first compartment, a hair treatment composition which comprises, in a cosmetically acceptable aqueous medium, at least one compositionally gradient copolymer as described below and, in a second compartment, a compressed gas chosen from air, nitrogen, carbon dioxide and their mixtures and optionally at least one liquefied gas.

Another subject-matter of the present invention is the use of at least one compositionally gradient copolymer as described below in two-compartment aerosol devices comprising a specific compressed gas as described below, optionally in combination with at least one liquefied gas, as propellant for the treatment and/or the shaping of the hairstyle.

Other characteristics, aspects and advantages of the invention will become even more clearly apparent on reading the description and various examples which follow.

According to the present invention, the two-compartment aerosol device comprises:
(a) in a first compartment, a hair treatment composition which comprises, in a cosmetically acceptable aqueous medium, at least one compositionally gradient copolymer comprising at least two different monomers and exhibiting a weight polydispersity index (PI) of less than or equal to 2.5, and
(b) in a second compartment, a compressed gas chosen from air, nitrogen, carbon dioxide and their mixtures, air being particularly preferred, and optionally at least one liquefied gas.

Said compressed gas is preferably used under a pressure of between 1 and 14 bar, better still of between 1 and 12 bar and more preferably still of between 9 and 11 bar.

Mention may in particular be made, as examples of liquefied gas, of hydrocarbons, such as C₁₋₅ alkanes, for example methane, propane, butane or pentane, and dimethyl ether.

The amount of liquefied gas is preferably between 0 and 95% by weight, better still between 0 and 60% by weight, with respect to the total weight of the propellant composed of the compressed and liquefied gases.

The particularly preferred propellant present in the second compartment is composed solely of at least one compressed gas.

The term “compositionally gradient copolymer” is understood to mean, within the meaning of the present invention, a copolymer having a distribution of at least one monomer of the polymer chains which changes in a given direction all along these chains and in a reproducible fashion from one chain to another.

The compositionally gradient copolymers used in the invention comprises at least two different monomers and exhibit a low dispersity in weight as well as, preferably, a low dispersity in composition.

A low dispersity in weight means that the lengths of chains are approximately identical.

The dispersity in weight can be represented using the weight polydispersity index (PI) of the copolymer, which is equal to the ratio of the weight-average molecular weight (Mw) to the number-average molecular weight (Mn).

The compositionally gradient copolymer used in the invention exhibits a weight polydispersity index of less than or equal to 2.5, preferably of between 1.1 and 2.3, better still between 1.15 and 2.0, more preferably between 1.2 and 1.9 or 1.8.

The weight-average molecular weight (Mw) of the gradient copolymer is preferably between 5000 and 1 000 000 g/mol, better still between 5500 and 800 000 g/mol and more preferably still between 6000 and 500 000 g/mol.

Preferably, the number-average molecular weight (Mn) of the compositionally gradient copolymer is between 5000 and 1 000 000 g/mol, better still between 5500 and 800 000 g/mol and more preferably still between 6000 and 500 000 g/mol.

The weight-average molecular weights (Mw) and the number-average molecular weights (Mn) can in particular be determined by gel permeation liquid chromatography (GPC) with a refractometric detector and tetra-hydrofuran (THF) as eluent, the calibrating curve being established with linear polystyrene standards.

The compositionally gradient copolymers used in the invention also preferably exhibit a low dispersity in composition. This means that all the chains of copolymers have a composition (that is to say, a sequence of monomers) which is approximately the same and are therefore homogeneous in composition.

In order to show that all the chains of copolymers have a similar composition, use may advantageously be made of liquid adsorption chromatography (or LAC), which makes it possible to separate the chains of copolymers not according to their molecular weight but according to their polarity. The latter makes it possible to determine the chemical composition of the polymers constituting the material, the monomers being known.

Reference may be made to the publication Macromolecules (2001), 34, 2667, which describes the LAC technique.

The polydispersity in composition can be defined in particular from the adsorption chromatography (LAC) curve, which is a curve representing the proportion of polymers as a function of the elution volume. If “V^{1/2} min” is used to denote the minimum value of the elution volume at mid-height of the curve and if “V^{1/2} max” is used to denote the maximum value of the elution volume at mid-height of the curve, the polydispersity in composition is regarded as low if the difference (V^{1/2} max−V^{1/2} min) is less than or equal to 3.5, preferably between 1 and 2.8 and better still between 1.2 and 2.5.

Furthermore, the LAC curve exhibits a gaussian curve profile and more particularly a gaussian curve profile defined by the formula:

$$y = \frac{A}{w \sqrt{\frac{π}{2}}} ⨯ {ⅇ}^{- 2 \frac{{( x - {x}_{0} )}^{2}}{{w}^{2}}} + {y}_{o}$$

in which:
x₀ represents the value of x (elution volume) at the center of the peak,
w is equal to twice the standard deviation of the gaussian distribution (i.e. 2σ) or alternatively corresponds to approximately 0.849 times the width of the peak at mid-height,
A represents the area under the peak,
yₒ represents the value of y corresponding to x₀.

The dispersity in composition can also be defined by the value w as defined above. Preferably, the said value w is between 1 and 3, better still between 1.1 and 2.3 and more preferably still between 1.1 and 2.0.

The gradient copolymers used in the invention can be obtained by living or pseudo-living polymerization.

Living polymerization is a polymerization in which the growth of the polymer chains only stops when the monomer disappears. The number-average molecular weight (Mn) increases with the conversion. Anionic polymerization is a typical example of living polymerization. Such polymerizations result in copolymers having a low dispersity in weight, that is to say in polymers with a weight polydispersity index (PI) generally of less than 2.

For its part, pseudo-living polymerization is associated with controlled radical polymerization. Mention may be made, among the main types of controlled radical polymerization, of:

radical polymerization controlled by nitroxides. Reference may in particular be made to patent applications WO 96/24620 and WO 00/71501, which disclose the devices of this polymerization and their use, and to the papers published by Fischer (Chemical Reviews, 2001, 101, 3581), by Tordo and Gnanou (J. Am. Chem. Soc., 2000, 122, 5929) and by Hawker (J. Am. Chem. Soc., 1999, 121, 3904);

atom transfer radical polymerization, disclosed in particular in application WO 96/30421 and which proceeds by the reversible insertion of an organo-metallic complex in a bond of carbon-halogen type;

radical polymerization controlled by sulfur derivatives of xanthate, dithioester, trithiocarbonate or dithiocarbamate type, such as disclosed in applications FR 2 821 620, WO 98/01478, WO 99/35177, WO 98/58974, WO 99/31144 and WO 97/01478 and in the publication by Rizzardo et al. (Macromolecules, 1998, 31, 5559).

Controlled radical polymerization denotes polymerizations in which the secondary reactions which usually result in the disappearance of propagating entities (termination or transfer reaction) are rendered highly improbable in comparison with the propagation reaction by virtue of an agent for controlling the free radicals. One disadvantage of this method of polymerization lies in the fact that, when the concentrations of free radicals become high in comparison with the concentration of monomer, the secondary reactions again become determining and tend to broaden the distribution of the masses.

By virtue of these polymerization methods, the polymer chains of the compositionally gradient copolymers used in the invention grow simultaneously and therefore incorporate at each instant the same ratios of comonomers. All the chains therefore have the same structures or similar structures, resulting in a low dispersity in composition. These chains also have a low weight polydispersity index.

In the case of conventional block polymers and random polymers, the change in the monomers along the polymer chain is not gradual and systematic.

As illustrated by the diagram below, a random polymer obtained by conventional radical polymerization of two monomers is distinguished from a compositionally gradient copolymer by the distribution of the monomers, which is not identical over all the chains, and by the length of the said chains, which is not identical for all the chains.

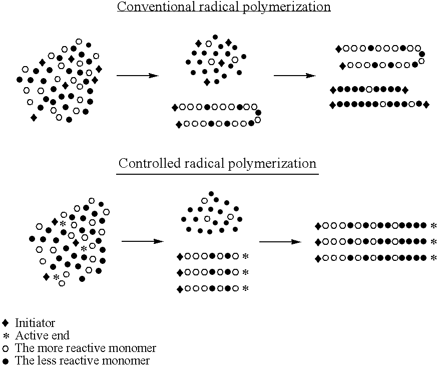

For a theoretical description of compositionally gradient copolymers, reference may be made to the following publications:
T. Pakula et al., Macromol. Theory Simul., 5, 987-1006 (1996);
A. Aksimetiev et al., J. of Chem. Physics, 111, No. 5;
M. Janco, J. Polym. Sci., Part A: Polym. Chem. (2000), 38(15), 2767-2778;
M. Zaremski et al., Macromolecules (2000), 33(12), 4365-4372;
K. Matyjaszewski et al., J. Phys. Org. Chem. (2000), 13(12), 775-786;
Gray, Polym. Prepr. (Am. Chem. Soc., Div. Polym. Chem.) (2001), 42(2), 337-338;
K. Matyjaszewski, Chem. Rev. (Washington, D.C.) (2001), 101(9), 2921-2990.

Among compositionally gradient copolymers, it is possible to distinguish natural gradient copolymers and artificial gradient copolymers.

A natural gradient copolymer is a compositionally gradient copolymer which can be obtained by batchwise synthesis from a starting mixture of comonomers. The distribution in the chain of the various monomers follows a law deduced from the relative reactivity and from the starting concentrations of monomers. These copolymers constitute the simplest class of compositionally gradient copolymers as it is the starting mixture which defines the final product property.

An artificial gradient copolymer is a copolymer for which the concentration of monomers during the synthesis can be varied by a processing expedient. In this case, a mixture of monomers is changed to another in the chain due to a sudden and abrupt change in the monomers in the reaction medium (for example, addition of at least one new monomer).

The gradient is characterized experimentally by measuring, during polymerization, the chemical composition of the polymer. This measurement is performed indirectly by determining the change in the concentration of the various monomers at any instant. It can be performed by NMR and UV spectroscopy, for example.

This is because, for the polymers prepared by living or pseudo-living polymerization, the length of the chains is linearly related to the conversion. By withdrawing a sample of the polymerization solution at various instants in the polymerization and by measuring the difference in content of each monomer, the composition of the gradient is thus determined.

The distribution of the compositions of the chains is narrow in the compositionally gradient polymer. In particular, there exists no overlap between the chromatographic peak of the compositionally gradient copolymer and those of the respective homopolymers. This means that the material obtained under gradient conditions is composed of polymer chains with the same composition whereas, in conventional random polymerization, different kinds of chain coexist, including those of the respective homopolymers.

It is possible to characterize gradient copolymers by a vector characteristic of each copolymer.

This is because, knowing that there exists an infinity of polymers characterized by a given chemical composition, to specify a polymer it is possible to describe the distribution of monomers along the chain. This involves a description comprising several variables. This vector is a point of the space of the chemical compositions.

The exact term is that G is a vector, the coordinates of which are the concentrations of the monomers along the polymer chain. These concentrations are defined by the rules of the reactivity coefficients of each of the monomers and therefore are related to the concentration of the free monomers during the synthesis: from the moment that the monomer is not in zero concentration in the reaction mixture, it is not in zero concentration in the polymer.

It is therefore possible to characterize compositionally gradient copolymers by the function G(x) which defines the composition gradient:
\vec{G}(x)=Σ\vec{[Mi](x)}
in which:
x denotes a normalized position on the polymer chain and
[Mi] (x) is the relative concentration, in this position x, of the monomer Mi, expressed in mol %.

The function G(x) therefore locally describes the composition of the gradient copolymer.

Two copolymers can have an equivalent composition overall but very different local distributions of the monomers and therefore different gradients.

The factors which determine the gradient are, first, the relative reactivity coefficients of each monomer (referred to as rᵢ for the monomer Mi), which depend mainly on the type of synthesis process employed (homogeneous, dispersed) and on the solvents and, secondly, the starting concentrations of each of the monomers and the possible additions of monomers during the polymerization.

The compositionally gradient copolymer used in the invention comprises at least two different monomers which can each be present in a proportion of 1 to 99% by weight, with respect to the final copolymer, in particular in a proportion of 2-98% by weight, preferably in a proportion of 5-95% by weight.

Preferably, at least one of the monomers of the compositionally gradient copolymer is a hydrophilic monomer.

In the present description, the term “hydrophilic monomer” is understood to mean monomers having homopolymers which are soluble or dispersible in water or an ionic form of which is soluble or dispersible in water.

A homopolymer is said to be water-soluble if it forms a clear solution when it is in solution at 5% by weight in water at 25° C.

A homopolymer is said to be water-dispersible if, at 5% by weight in water at 25° C., it forms a stable suspension of fine, generally spherical, particles. The mean size of the particles constituting said dispersion is less than 1 μm and more generally varies between 5 and 400 nm, preferably from 10 to 250 nm. These particle sizes are measured by light scattering.

Preferably, the hydrophilic monomer exhibits a glass transition temperature (hereinafter denoted Tg) of greater than or equal to 20° C., better still of greater than or equal to 50° C., but can optionally have a Tg of less than or equal to 20° C.

The glass transition temperature (or Tg) can be measured according to Standard ASTM D 3418-97 by Differential Scanning Calorimetry (DSC) with a calorimeter over a temperature range of between −100° C. and +150° C. at a heating rate of 10° C./min in 150 μl aluminum crucibles.

Mention may be made, among the hydrophilic monomers capable of being employed in the present invention, of the following monomers:
amino(C₁-C₄ alkyl) (meth)acrylate derivatives and in particular N,N-di(C₁-C₄ alkyl)amino(C₁-C₆ alkyl) (meth)acrylates, such as N,N-dimethylaminoethyl methacrylate (MADAME) or N,N-diethylaminoethyl methacrylate (DEAMEA);
di(C₁-C₈ alkyl)allylamines, such as dimethylallylamine;
vinylamine;
vinylpyridines, in particular 2-vinylpyridine or 4-vinylpyridine;
   and their salts with inorganic acids or with organic acids or their quaternized forms.

Mention may in particular be made, among inorganic acids, of sulfuric acid, hydrochloric acid, hydrobromic acid, hydriodic acid, acetic acid, propionic acid, phosphoric acid or boric acid.

Mention may be made, among organic acids, for example, of acids comprising one or more carboxyl, sulfo or phosphonic groups. They can be linear, branched or cyclic aliphatic acids or aromatic acids. These acids can additionally comprise one or more heteroatoms chosen from O and N, for example in the form of hydroxyl groups.

Mention may be made, as examples of organic acids, of acids comprising an alkyl group, such as acetic acid CH₃COOH, polyacids, such as terephthalic acid, and hydroxy acids, such as citric acid and tartaric acid.

The quaternizing agents can be alkyl halides, such as methyl bromide, or alkyl sulfates, such as methyl sulfate, or propane sultone.

Mention may also be made, as examples of hydrophilic monomer, of:
ethylenic carboxylic acids, in particular mono- or dicarboxylic acids, comprising from 3 to 20 carbon atoms, or their salts, such as acrylic acid, methacrylic acid, crotonic acid, itaconic acid, fumaric acid, maleic acid and vinylbenzoic acid;
carboxylic anhydrides carrying a vinyl double bond and comprising from 4 to 30 carbon atoms, such as maleic anhydride;
ethylenic sulfonic or phosphonic acids, or their salts, such as styrenesulfonic acid, acrylamidopropanesulfonic acid, vinylphosphonic acid and their salts, the potassium salt of acryloyloxy-3-sulfopropyl, or the compound of formula CH₂═CHCOOCH₂OCH₂(OH)CH₂SO₃⁻Na⁺,
vinyl alcohol.

The neutralizing agent can be an inorganic base, such as LiOH, NaOH, KOH, Ca(OH)₂ or NH₄OH, or an organic base, for example a primary, secondary or tertiary amine, in particular an alkylamine, which is optionally hydroxylated, such as dibutylamine, triethylamine, stearamine, or else 2-amino-2-methylpropanol, monoethanolamine, diethanolamine or stearamidopropyldimethylamine.

Mention may also be made, as examples of hydrophilic monomer, of:
unsaturated carboxamides, such as acrylamide or methacrylamide, and their N-substituted or N,N-di-substituted analogs, such as N—(C₁-C₆ alkyl) (meth)acrylamides, for example N-methylacrylamide, N-isopropylamide, N-butylamide and N-(tert-butyl)amide, and more particularly N—(C₁-C₃ alkyl) (meth)acrylamides, such as N-methylacrylamide; N,N-di(C₁-C₃ alkyl) (meth)acrylamides, such as N,N-dimethylacrylamide; or N,N-di(C₁-C₄ alkyl)-amino(C₁-C₆ alkyl) (meth)acrylamides, such as N,N-dimethylaminopropylacrylamide (DMAPA) or N,N-dimethylaminopropylmethacrylamide (DMAPMA);
hydroxyalkyl (meth)acrylates, in particular those having an alkyl group comprising from 2 to 4 carbon atoms, in particular hydroxyethyl (meth)acrylate;
polyethylene glycol (5 to 100 ethylene oxide or EO units) or glycol (meth)acrylates which are or are not substituted on their terminal functional group by C₁-C₄ alkyl, phosphate, phosphonate or sulfonate groups, for example glyceryl acrylate, methoxypolyethylene glycol (8 or 12 EO) (meth)acrylate or hydroxypolyethylene glycol (meth)acrylate;
C₁-C₄ alkoxyalkyl. (meth)acrylates, such as methoxyethyl or ethoxyethyl (meth)acrylate;
polysaccharide (meth)acrylates, such as sucrose acrylates;
vinylamides, such as N-vinylacetamide, which are optionally cyclic, such as, in particular, vinyllactams, such as N-vinylpyrrolidone or N-vinylcaprolactam;
vinyl ethers, such as ethers of vinyl and of alkyl having 1 to 12 carbon atoms, such as methyl vinyl ether and ethyl vinyl ether.

Mention may also be made, as examples of hydrophilic polymer, of the following compounds of betaine type:
methacrylamidopropoxytrimethylammonium;
N,N-dimethyl-N-methacryloyloxyethyl-N-(3-sulfopropyl)ammonium;
3-methacryloylethoxycarbonylpyridinium,
the compound of formula: 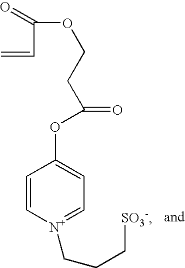
N-(3-sulfopropyl)-4-vinylpyridinium of formula:

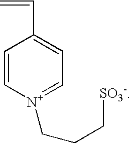

At least one of the monomers of the compositionally gradient copolymer can also be a hydrophobic monomer, in particular a hydrophobic monomer capable of being rendered hydrophilic after polymerization, or a mixture of such monomers. The hydrophobic monomer(s) can be rendered hydrophilic, for example, by chemical reaction, in particular by hydrolysis, or by chemical modification, in particular of an ester functional group, by incorporation of chains comprising a hydrophilic unit, for example of carboxylic acid type.

Preferably, the hydrophilic monomers are chosen from N,N-dimethylaminoethyl methacrylate (MADAME), acrylic acid, methacrylic acid, crotonic acid, styrenesulfonic acid, acrylamidopropanesulfonic acid, dimethylaminopropylmethacrylamide (DMAPMA), styrenesulfonate, hydroxyethyl acrylate, glyceryl acrylate, methoxyethyl (meth)acrylate, ethoxyethyl (meth)acrylate, methoxypolyethylene glycol (8 or 12 EO) (meth)acrylate, hydroxypolyethylene glycol (meth)acrylate, N-vinylpyrrolidone, N-vinylcaprolactam, acrylamide or N,N-dimethylacrylamide.

The hydrophilic monomer or monomers can be present in a proportion of 1 to 99% by weight, preferably of 2 to 70% by weight, better still of 5 to 50% by weight, more preferably still of 10 to 30% by weight, with respect to the total weight of the copolymer.

At least one of the monomers of the compositionally gradient copolymer used in the invention can be, preferably, a hydrophobic monomer.

Mention may be made, among hydrophobic monomers capable of being employed in the present invention, of:
ethylenic hydrocarbons comprising from 2 to 30 carbon atoms, such as ethylene, isoprene and butadiene;
acrylates of formula CH₂═CHCOOR₁, in which R₁ represents a saturated or unsaturated, linear, branched or cyclic, hydrocarbon group comprising from 1 to 30 carbon atoms in which one or more heteroatoms chosen from O, N, S and Si is/are optionally inserted, it being possible for said hydrocarbon group additionally to be optionally substituted by one or more substituents chosen from hydroxyl groups and halogen atoms (Cl, Br, I and F).

Mention may in particular be made, as examples of a hydrocarbon group for R₁, of a C₁-C₃₀ alkyl group, it being possible for said alkyl group also to be optionally substituted by one or more substituents comprising Si; a C₃-C₈ cycloalkyl group; a C₆ to C₂₀ aryl group; a C₇ to C₃₀ aralkyl group (C₁ to C₄ alkyl group); or a 4- to 12-membered heterocyclic group comprising one or more heteroatoms chosen from O, N and S; it being possible for said cycloalkyl, aryl, aralkyl and heterocyclic groups also to be optionally substituted by one or more linear or branched alkyl groups having from 1 to 4 carbon atoms in which one or more heteroatoms chosen from O, N, S and P is/are optionally inserted, it being possible for said alkyl groups additionally to be optionally substituted by one or more substituents chosen from hydroxyl groups and halogen (Cl, Br, I and F) or Si atoms.

Mention may in particular be made, as preferred examples of such R₁ groups, of the methyl, ethyl, propyl, butyl, isobutyl, tert-butyl, hexyl, ethylhexyl, octyl, lauryl, isooctyl, isodecyl, t-butylcyclohexyl, t-butylbenzyl, furfuryl, isobornyl, ethylperfluorooctyl and propylpolydimethylsiloxyl groups.

R₁ can also be a —(R₁₀)ₓ—(OC₂H₄)ₙ—OR₁₁ group, with x=0 or 1, R₁₀=saturated or unsaturated, linear or branched, divalent hydrocarbon group, such as alkylene or alkenylene, comprising from 1 to 30 carbon atoms, n=5 to 100 and R₁₁=H or CH₃; and in particular a methoxy(PEO)₈stearyl group with PEO=poly(ethylene oxide).
methacrylates of formula CH₂═C(CH₃)—COOR₂, in which R₂ represents a saturated or unsaturated, linear, branched or cyclic, hydrocarbon group comprising from 1 to 30 carbon atoms in which one or more heteroatoms chosen from O, N, S and Si is/are optionally inserted, it being possible for said hydrocarbon group additionally to be optionally substituted by one or more substituents chosen from hydroxyl groups and halogen atoms (Cl, Br, I, F).

Mention may in particular be made, as examples of hydrocarbon groups for R₂, of a linear or branched alkyl group having from 1 to 30 carbon atoms, it being possible for said alkyl group also to be optionally substituted by one or more substituents comprising Si; a C₃ to C₈ cycloalkyl group; a C₆ to C₂₀ aryl group; a C₇ to C₃₀ aralkyl group (C₁ to C₄ alkyl group); or a 4- to 12-membered heterocyclic group comprising one or more heteroatoms chosen from O, N and S; it being possible for said cycloalkyl, aryl, aralkyl and heterocyclic groups also to be optionally substituted by one or more linear or branched alkyl groups having from 1 to 4 carbon atoms in which one or more heteroatoms chosen from O, N, S and P is/are optionally inserted, it being possible for said alkyl groups additionally to be optionally substituted by one or more substituents chosen from hydroxyl groups and halogen atoms (Cl, Br, I and F).

Preferred examples of R₂ groups are the methyl, ethyl, propyl, n-butyl, isobutyl, hexyl, ethylhexyl, octyl, lauryl, isooctyl, isodecyl, dodecyl, tert-butylcyclohexyl, isobornyl, tert-butylbenzyl, ethylperfluorooctyl and propylpolydimethylsiloxyl groups;

R₂ can also be a —(R₁₀)ₓ—(OC₂H₄)ₙ—OR₁₁ group, with x=0 or 1, R₁₀=saturated or unsaturated, linear or branched, divalent hydrocarbon group, such as alkylene or alkenylene, comprising from 1 to 30 carbon atoms, n=5 to 100 and R₁₁=H or CH₃; and in particular a methoxy(PEO)₈stearyl group.

The examples of methacrylate monomers are methyl, ethyl, n-butyl, isobutyl, t-butylcyclohexyl, t-butylbenzyl and isobornyl methacrylates.
N-substituted or N,N-disubstituted unsaturated carboxamides, such as N—(C₈₋₃₀ alkyl) (meth)acrylamides, for example N-octylacrylamide;
vinyl esters of formula R₃—CO—O—CH═CH₂, where R₃ represents a linear or branched alkyl group having from 2 to 30 carbon atoms, in particular vinyl propionate, vinyl butyrate, vinyl ethylhexanoate, vinyl neononanoate and vinyl neododecanoate;
vinyl compounds of formula CH₂═CH—R₄, where R₄ is an —OC(O)—CH₃ group, a C₃ to CB cycloalkyl group, a C₆ to C₂₀ aryl group, a C₇ to C₃₀ aralkyl group (C₁ to C₄ alkyl group) or a 4- to 12-membered heterocyclic group comprising one or more heteroatoms chosen from O, N and S, it being possible for said cycloalkyl, aryl, aralkyl and heterocyclic groups to be optionally substituted by one or more substituents chosen from hydroxyl groups, halogen atoms and linear or branched alkyl groups having from 1 to 4 carbon atoms in which one or more heteroatoms chosen from O, N, S and P is/are optionally inserted, it being possible for said alkyl groups additionally to be optionally substituted by one or more substituents chosen from hydroxyl groups and halogen (Cl, Br, I and F) or Si atoms.

Examples of such vinyl monomers are vinylcyclohexane, styrene and vinyl acetate.

Preferably, the hydrophobic monomers are chosen from:
isoprene and butadiene;
methyl, ethyl, isobutyl, n-butyl, tert-butyl, ethylhexyl, furfuryl, isobornyl, tert-butylcyclohexyl or tert-butylbenzyl acrylates;
methyl, ethyl, n-butyl, isobutyl, hexyl or ethylhexyl methacrylates;
N—(C₈₋₁₂ alkyl) (meth)acrylamides, such as N-octylacrylamide;
vinyl esters of formula R₃—CO—O—CH═CH₂, where R₃ represents a linear or branched alkyl group having from 6 to 30 carbon atoms, in particular vinyl neononanoate and vinyl neododecanoate;
styrene;
vinyl acetate and vinylcyclohexane.

These monomers can be present in a proportion of 1 to 99% by weight, preferably of 10 to 90% by weight, better still of 20 to 80% by weight, more preferably still of 25 to 75% by weight, with respect to the total weight of the copolymer.

In a preferred embodiment, the compositionally gradient copolymer used in the invention comprises three different monomers which can be present in a proportion of 5-90% by weight each, preferably 7-86% by weight each, with respect to the total weight of the copolymer.

In particular, the copolymer can comprise 5-25% by weight of a first monomer, 5-25% by weight of a second monomer and 50-90% by weight of a third monomer.

Preferably, the copolymer according to the invention can comprise 5-25% by weight of a hydrophilic monomer, 50-90% by weight of a monomer with a Tg of less than or equal to 20° C. and 5-25% by weight of an additional monomer.

A person skilled in the art will know how to choose the monomers and their amounts according to the results desired, taking as basis his general knowledge, in particular his knowledge of the relative reactivity of each monomer.

Thus, if a copolymer having hydrophilic units in the heart of a polymer chain is desired, a difunctional initiator and a mixture of monomers such that the reactivity of the hydrophilic monomers is greater than that of the other monomers will preferably be chosen.

Furthermore, it has been found that the preparation processes employed make it possible to adjust and modify the Tg value or values of the copolymer and thus to obtain a compositionally gradient copolymer having one or more given Tg value(s).

The compositionally gradient copolymers used in the invention can be prepared by a person skilled in the art according to the following procedure:

1) A mixture of the various monomers is prepared, optionally in a solvent, preferably in a reactor and with stirring. A radical polymerization initiator and an agent for controlling the polymerization are added. The mixture is preferably placed under a gas atmosphere which is inert with respect to radical polymerization, such as nitrogen or argon.

The choice may be made, as optional polymerization solvent, of alkyl acetates, such as butyl acetate or ethyl acetate, aromatic solvents, such as toluene, ketone solvents, such as methyl ethyl ketone, or alcohols, such as ethanol. In the case where the mixture of monomers is miscible with water, the latter can advantageously be used as solvent or cosolvent.

2) The mixture is brought with stirring to the desired polymerization temperature. This temperature is preferably chosen within a range from 10° C. to 160° C., more preferably from 25° C. to 130° C.

The choice of the polymerization temperature is preferably optimized according to the chemical composition of the mixture of monomers. Thus, monomers having very high propagation kinetic constants and a weaker affinity for the control agent will preferably be polymerized at low temperature (for example, in the case of a high proportion of methacrylic derivatives, polymerization at a temperature of between 25° and 80° C. will be preferred).

3) The polymerization medium is optionally modified during the polymerization, before 90% conversion of the starting monomers is achieved, by further addition of one or more monomers, in particular of the starting mixture. This addition can be carried out in various ways, which can range from the sudden addition all at once to the continuous addition over the entire duration of the polymerization.

4) The polymerization is halted when the desired degree of conversion is achieved. The overall composition of the copolymer depends on this conversion. The polymerization is preferably halted after having achieved at least 50% conversion, in particular at least 60%, preferably after having achieved at least 90% conversion.

5) The possible residual monomers can be removed by any known method, such as by evaporation or by addition of an amount of conventional polymerization initiator, such as peroxide or azo derivatives.

In a first embodiment, the agent for controlling the polymerization capable of being employed is a nitroxide of formula (I), alone or as a mixture: 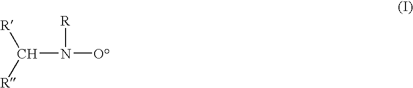
in which:

R and R′ are, independently of one another, linear or branched, saturated hydrocarbon (alkyl) groups comprising 1 to 40 carbon atoms which are optionally substituted by one or more groups chosen from —OR₄, —COOR₄ and —NHR₄ (with R₄ representing H or a linear or branched, saturated hydrocarbon (alkyl) group comprising 1 to 40 carbon atoms), it being possible in addition for R and R′ to be connected so as to form a ring.

In particular, R and R′ are linear or branched alkyl groups comprising 1 to 12 carbon atoms, in particular methyl, ethyl, propyl, n-butyl, isobutyl, tert-butyl or pentyl groups. Preferably, R and R′ are both tert-butyl groups;

R″ is a monovalent group with a molar mass (Mw) of greater than 16 g/mol, in particular a phosphorus-comprising group of formula: 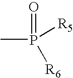
in which R₅ and R₆ are, independently of one another, linear or branched, saturated hydrocarbon, preferably alkyl, groups comprising 1 to 40 carbon atoms which are optionally substituted by one or more groups chosen from —OR₄, —COOR₄ and —NHR₄ (with R₄ representing H or a linear or branched, saturated hydrocarbon, preferably alkyl, group comprising 1 to 40 carbon atoms), it being possible in addition for R₅ and R₆ to be connected so as to form a ring.

In particular, R₅ and R₆ are linear or branched alkyl groups comprising 1 to 12 carbon atoms, in particular methyl, ethyl, propyl, n-butyl, isobutyl, tert-butyl or pentyl groups. Preferably, R₅ and R₆ are both ethyl groups.

The radical polymerization initiator can be chosen from any conventional polymerization initiator, such as compounds of azo type, and in particular azobis(isobutyronitrile), or of peroxide type, such as organic peroxides having 6-30 carbon atoms, in particular benzoyl peroxide.

Preferably, a nitroxide/initiator molar ratio of between 1 and 2.5 is observed; this ratio can be between 2 and 2.5 when it is considered that one mole of initiator gives rise to two moles of polymer chains and can be between 1 and 1.25 for monofunctional initiators.

In a second specific embodiment, it is possible to employ, as radical polymerization initiator, alkoxyamines of formula (II) 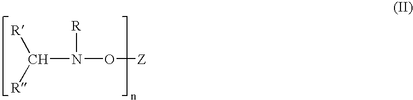
in which:
R, R′ and R″ have the meanings given above,
n is an integer of less than or equal to 8, preferably of between 1 and 3;
Z is a monovalent or polyvalent radical, in particular a styryl, acryloyl or methacryloyl radical, which can advantageously be chosen in order to initiate the polymerization and, at the same time, release the nitroxide which controls this polymerization.

A nitroxide of formula (I) can also be added to the alkoxyamine of formula (II) in a proportion ranging from 0 to 20 mol % with respect to the numbers of moles of alkoxyamine functional groups (one mole of polyvalent alkoxyamine contributes a number of alkoxyamine functional groups proportional to its valency), so as to improve the quality of the polymerization control.

A person skilled in the art will know how to choose the initiator according to the requirements of the application. Thus, a monofunctional initiator will result in asymmetric chains, whereas a polyfunctional initiator will result in macromolecules having a symmetry starting from a core.

The copolymers can be present in the composition in the dissolved form, for example dissolved in water or an organic solvent, or else in the form of an aqueous or organic dispersion.

It is possible to prepare an aqueous solution of the copolymer by directly mixing the polymer with water, optionally while heating.

It is also possible to dissolve the polymer in an organic solvent with a lower boiling point than water (for example, acetone or methyl ethyl ketone), at a level of solid of between 20 and 90% by weight.

When the hydrophilic monomers are of acid type, a solution, preferably of at least 1 M, of base, such as a hydroxonium ion (OH⁻) salt, an amine (ammonia), a carbonate (CO₃²⁻) salt or a hydrogencarbonate (HCO₃⁻) salt, or of organic neutralizing agent can be added to the organic solution. In the case of hydrophilic monomers of amine type, a solution, preferably at least 1 M, of acid can be added. Water is then added to the solution with vigorous stirring in a proportion such that the level of solid obtained is between 1 and 80% by weight. The water can optionally be replaced by an aqueous/alcoholic mixture in proportions ranging from 99/1 to 50/50. The solvent is evaporated while stirring the solution at 100° C. Concentrating is continued until the desired level of solid is obtained.

The compositionally gradient copolymer(s) used in the context of the present invention is or are generally present in an amount ranging from 0.1 to 20% by weight, preferably ranging from 1 to 17% by weight, better still ranging from 5 to 15% by weight, with respect to the total weight of the hair treatment composition.

The term “cosmetically acceptable medium” is understood to mean any medium compatible with keratinous substances and in particular with hair.

The cosmetically acceptable aqueous medium comprises water and/or one or more cosmetically acceptable solvents. The cosmetically acceptable solvents are chosen in particular from lower C₁-C₄ alcohols, such as ethanol, isopropanol, tert-butanol or n-butanol, polyols, such as propylene glycol, and polyol ethers, acetone, and their mixtures. The particularly preferred solvent being ethanol.

The proportion of water can be between 80 and 99.9% by weight, preferably between 95 and 97% by weight, with respect to the total weight of the hair treatment composition. Advantageously, the medium is aqueous or a water/alcohol mixture. When the alcohol is present, its proportion in the mixture is in particular between 1 and 99% by weight, preferably between 5 and 80% by weight and more preferably still between 8 and 50% by weight, with respect to the total weight of the hair treatment composition.

The hair treatment composition according to the invention can additionally comprise at least one adjuvant chosen from silicones in the soluble, dispersed or microdispersed form, nonionic, anionic, cationic and amphoteric surface-active agents, ceramides and pseudoceramides, vitamins and provitamins, including panthenol, vegetable, animal, mineral and synthetic oils, waxes, water-soluble and fat-soluble sunscreens which may or may not comprise a silicone portion, colored or colorless inorganic and organic pigments, dyes, pearlescent and opacifying agents, sequestering agents, plasticizing agents, solubilizing agents, acidifying agents, basifying agents, inorganic and organic thickening agents, antioxidants, hydroxy acids, penetrating agents, fragrances and preservatives.

A person skilled in the art will take care to choose the optional additives and their amounts so that they do not interfere with the properties of the compositions of the present invention.

These additives are present in the composition according to the invention in an amount ranging from 0 to 20% by weight, with respect to the total weight of the composition.

The hair treatment compositions present in the device according to the invention can be used for the shaping and/or the form retention of the hairstyle, for example as compositions for the fixing and/or form retention of the hair, hair care compositions, shampoos, hair conditioning compositions, such as compositions intended to contribute softness to the hair, or hair make-up compositions.

Preferably, the two-compartment aerosol device is composed of an external aerosol can comprising an internal bag hermetically welded to a valve. The composition is introduced into the internal bag and a compressed gas is introduced between the bag and the can at a pressure sufficient to bring about the departure of the product in the form of a spray through the orifice of a nozzle. Such a device is sold under the name EP Spray by EP-Spray System S.A.

More particularly, the present invention also relates to the use of the product vaporized by the aerosol device according to the invention for the shaping and/or the form retention of the hairstyle, for example as hair lacquer.

The present invention also relates to a styling process comprising the stage consisting in vaporizing, over wet or dry hair, the hair treatment composition present in the aerosol device according to the invention.

The following examples are given by way of illustration of the present invention.

### EXAMPLES

### Example 1

The following composition was prepared by mixing the ingredients indicated below.

| | Amount |
|---|---|
| | (% by weight) |
| Gradient copolymer: poly(methacrylic | 5 |
| acid/styrene)/poly(butyl acrylate) | |
| Water | 95 |

The composition prepared above was introduced into the aerosol dispensing device described above sold under the name EP Spray by EP Spray System S.A. A valve with the reference 6001 format D6 is attached to a conventional aerosol can and the diffuser is a diffuser with a swirl-inducing nozzle.

The bag is filled with the composition as indicated above. Compressed air is introduced between the bag and the can.

The application of this product to dry or wet hair makes it possible to obtain good fixing without bleaching of the hair.

### Example 2

| | Amount |
|---|---|
| | (% by weight) |
| Gradient copolymer: poly(methacrylic | 5 |
| acid/styrene)/poly(ethyl acrylate) | |
| Water | 95 |

The operation was carried out as in example 1.

The application of this product to dry or wet hair makes it possible to obtain good fixing without bleaching of the hair.

### Example 3

| | Amount |
|---|---|
| | (% by weight) |
| Gradient copolymer: poly(methacrylic | 5 |
| acid/styrene)/poly(methyl acrylate/butyl | |
| acrylate) | |
| Water | 95 |

The operation was carried out as in example 1.

The application of this product to dry or wet hair makes it possible to obtain good fixing without bleaching of the hair.

What is claimed is:

1. A two-compartment aerosol device comprising:
(a) in a first compartment, a hair treatment composition which comprises, in a cosmetically acceptable aqueous medium, at least one compositionally gradient copolymer comprising at least two different monomers and exhibiting a weight polydispersity index (PI) of less than or equal to 2.5, and
(b) in a second compartment, a compressed gas chosen from air, nitrogen, carbon dioxide and their mixtures, and optionally at least one liquefied gas.

2. The aerosol device as claimed in claim 1, characterized in that the compressed gas is air.

3. The aerosol device as claimed in claim 1 or 2, characterized in that the pressure of the compressed gas is between 1 and 14 bar.

4. The aerosol device as claimed in claim 3, characterized in that the pressure of the compressed gas is between 9 and 11 bar.

5. The aerosol device as claimed in any one of the preceding claims, characterized in that the liquefied gas is chosen from hydrocarbons and dimethyl ether.

6. The aerosol device as claimed in claim 5, characterized in that the hydrocarbons are chosen from C₁₋₅ alkanes.

7. The aerosol device as claimed in any one of the preceding claims, characterized in that the liquefied gas is present in an amount ranging from 0 to 95% by weight, preferably between 0 and 60% by weight, with respect to the total weight of the propellant composed of the compressed and liquefied gases.

8. The aerosol device as claimed in any one of the preceding claims, characterized in that the weight polydispersity index (PI) is between 1.1 and 2.3.

9. The aerosol device as claimed in any one of the preceding claims, characterized in that the weight-average molecular weight of the compositionally gradient copolymer is between 5000 and 1 000 000 g/mol.

10. The aerosol device as claimed in any one of the preceding claims, characterized in that the number-average molecular weight of the compositionally gradient copolymer is between 5000 and 1 000 000 g/mol.

11. The aerosol device as claimed in any one of the preceding claims, characterized in that the compositionally gradient copolymer is such that, on the adsorption chromatography (LAC) curve representing the proportion of polymers as a function of the elution volume, the difference (V^{1/2} max−V^{1/2} min) is less than or equal to 3.5, preferably between 1 and 2.8, “V^{1/2} min” being the minimum value of the elution volume at mid-height of the curve and “V^{1/2} max” being the maximum value of the elution volume at mid-height of the curve.

12. The aerosol device as claimed in any one of the preceding claims, characterized in that at least one of the monomers of the compositionally gradient copolymer is a hydrophilic monomer.

13. The aerosol device as claimed in claim 12, characterized in that the hydrophilic monomer is chosen from:
amino(C₁-C₄ alkyl) (meth)acrylate derivatives;
di(C₁-C₈ alkyl)allylamines;
vinylamine;
vinylpyridines;
and their salts with inorganic acids or with organic acids, or their quaternized forms;
ethylenic carboxylic acids comprising from 3 to 20 carbon atoms, or their salts;
carboxylic anhydrides carrying a vinyl double bond and comprising from 4 to 30 carbon atoms;
ethylenic sulfonic or phosphonic acids, and their salts;
vinyl alcohol;
acrylamide or methacrylamide, N—(C₁-C₆ alkyl) (meth)-acrylamides, N,N-di(C₁-C₃ alkyl) (meth)acrylamides or N,N-di(C₁-C₄ alkyl)amino(C₁-C₆ alkyl) (meth)acrylamides;
hydroxy(C₂-C₄ alkyl) (meth)acrylates;
polyethylene glycol (5 to 100 ethylene oxide units) or glycol (meth)acrylates which are or are not substituted on their terminal functional group by C₁₋₄ alkyl, phosphate, phosphonate or sulfonate groups;
C₁₋₄ alkoxyalkyl (meth)acrylates;
polysaccharide (meth)acrylates;
optionally cyclic vinylamides;
vinyl ethers;
methacrylamidopropoxytrimethylammonium;
N,N-dimethyl-N-methacryloyloxyethyl-N-(3-sulfopropyl)-ammonium;
3-methacryloylethoxycarbonylpyridinium;
the compound of formula: 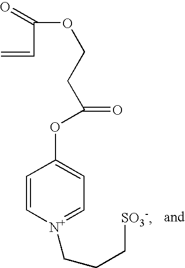
N-(3-sulfopropyl)-4-vinylpyridinium of formula:

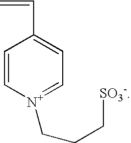

14. The aerosol device as claimed in claim 13, characterized in that the hydrophilic monomer is chosen from N,N-dimethylaminoethyl methacrylate (MADAME), acrylic acid, methacrylic acid, crotonic acid, styrenesulfonic acid, acrylamidopropanesulfonic acid, dimethylaminopropylmethacrylamide (DMAPMA), styrene-sulfonate, hydroxyethyl acrylate, glyceryl acrylate, methoxyethyl (meth)acrylate, ethoxyethyl (meth)acrylate, methoxypolyethylene glycol (8 or 12 EO) (meth)acrylate, hydroxypolyethylene glycol (meth)acrylate, N-vinyl-pyrrolidone, N-vinylcaprolactam, acrylamide and N,N-dimethylacrylamide.

15. The aerosol device as claimed in any one of the preceding claims, characterized in that at least one of the monomers of the compositionally gradient copolymer is a hydrophobic monomer.

16. The aerosol device as claimed in claim 15, characterized in that the hydrophobic monomer is chosen from:
ethylenic hydrocarbons comprising from 2 to 30 carbon atoms;
acrylates of formula CH₂═CHCOOR₁, in which R₁ represents a saturated or unsaturated, linear, branched or cyclic, hydrocarbon group comprising from 1 to 30 carbon atoms in which one or more heteroatoms chosen from O, N, S and Si is/are optionally inserted, said hydrocarbon group additionally being optionally substituted by one or more substituents chosen from hydroxyl groups and halogen atoms,
or R₁ represents a —(R₁₀)ₓ—(OC₂H₄)ₙ—OR₁₁ group, with x=0 or 1, R₁₀=saturated or unsaturated, linear or branched, divalent hydrocarbon group comprising from 1 to 30 carbon atoms, n=5 to 100 and R₁₁=H or CH₃;
methacrylates of formula CH₂═C(CH₃)—COOR₂, in which R₂ represents a saturated or unsaturated, linear, branched or cyclic, hydrocarbon group comprising from 1 to 30 carbon atoms in which one or more heteroatoms chosen from O, N, S and Si is/are optionally inserted, said hydrocarbon group additionally being optionally substituted by one or more substituents chosen from hydroxyl groups and halogen atoms,
or R₂ represents —(R₁₀)ₓ—(OC₂H₄)ₙ—OR₁₁, with x=0 or 1, R₁₀=saturated or unsaturated, linear or branched, divalent hydrocarbon group comprising from 1 to 30 carbon atoms, n=5 to 100 and R₁₁=H or CH₃;
N—(C₈₋₃₀ alkyl) (meth)acrylamides;
vinyl esters of formula R₃—CO—O—CH═CH₂, where R₃ represents a linear or branched alkyl group having from 2 to 12 carbon atoms;
vinyl compounds of formula CH₂═CH—R₄, where R₄ is an —OC(O)—CH₃ group, a C₃ to C₈ cycloalkyl group, a C₆ to C₂₀ aryl group, a C₇ to C₃₀ aralkyl group (C₁ to C₄ alkyl group) or a 4- to 12-membered heterocyclic group comprising one or more heteroatoms chosen from O, N and S, said cycloalkyl, aryl, aralkyl and heterocyclic groups optionally being substituted by one or more substituents chosen from hydroxyl groups, halogen atoms and linear or branched alkyl groups having from 1 to 4 carbon atoms in which one or more heteroatoms chosen from O, N, S and P is/are optionally inserted and said alkyl groups additionally being optionally substituted by one or more substituents chosen from hydroxyl groups and halogen or Si atoms.

17. The aerosol device as claimed in claim 16, characterized in that the hydrophobic monomer is chosen from:
isoprene and butadiene;
methyl, ethyl, isobutyl, n-butyl, tert-butyl, ethylhexyl, furfuryl, isobornyl, tert-butylcyclohexyl or tert-butylbenzyl acrylates;
methyl, ethyl, n-butyl, isobutyl, hexyl or ethylhexyl methacrylates;
N—(C₆₋₁₂ alkyl)(meth)acrylamides;
vinyl esters of formula R₃—CO—O—CH═CH₂, where R₃ represents a linear or branched alkyl group having from 6 to 12 carbon atoms;
styrene;
vinyl acetate and vinylcyclohexane.

18. The aerosol device as claimed in any one of the preceding claims, characterized in that the compositionally gradient copolymer(s) is or are present in an amount ranging from 0.1 to 20% by weight, preferably from 1 to 17% by weight, with respect to the total weight of the hair treatment composition.

19. The aerosol device as claimed in any one of the preceding claims, characterized in that the compositionally gradient copolymer is present in the dissolved form or else in the form of an aqueous or organic dispersion.

20. The aerosol device as claimed in any one of the preceding claims, characterized in that the cosmetically acceptable aqueous medium comprises water and/or one or more cosmetically acceptable solvents.

21. The aerosol device as claimed in claim 20, characterized in that the cosmetically acceptable solvent(s) is or are chosen from lower C₁-C₄ alcohols, polyols, polyol ethers, acetone and their mixtures.

22. The aerosol device as claimed in any one of the preceding claims, characterized in that water is present in an amount of between 80 and 99.9% by weight, preferably between 95 and 97% by weight, with respect to the total weight of the hair treatment composition.

23. The aerosol device as claimed in any one of the preceding claims, characterized in that the hair treatment composition additionally comprises at least one adjuvant chosen from silicones in the soluble, dispersed or microdispersed form, nonionic, anionic, cationic and amphoteric surface-active agents, ceramides and pseudoceramides, vitamins and provitamins, including panthenol, vegetable, animal, mineral and synthetic oils, waxes, water-soluble and fat-soluble sunscreens which may or may not comprise a silicone portion, colored or colorless inorganic and organic pigments, dyes, pearlescent and opacifying agents, sequestering agents, plasticizing agents, solubilizing agents, acidifying agents, basifying agents, inorganic and organic thickening agents, antioxidants, hydroxy acids, penetrating agents, fragrances and preservatives.

24. The use of the product vaporized by the aerosol device as claimed in any one of claims 1 to 23 for the shaping and/or the form retention of the hairstyle.

25. A styling process comprising the stage which consists in vaporizing, over wet or dry hair, the hair treatment composition present in the aerosol device as claimed in any one of claims 1 to 23.

26. The use of at least one compositionally gradient copolymer comprising at least two different monomers and exhibiting a weight polydispersity index (PI) of less than or equal to 2.5 in a two-compartment aerosol device comprising a compressed gas chosen from air, nitrogen, carbon dioxide and their mixtures, and optionally a liquefied gas, as propellant for the treatment and/or the shaping of the hairstyle.

27. The use as claimed in claim 26, characterized in that the compressed gas is air.

28. The use as claimed in claim 26 or 27, characterized in that the pressure of the compressed gas is between 1 and 14 bar.

29. The use as claimed in claim 28, characterized in that the pressure of the compressed gas is between 9 and 11 bar.

30. The use as claimed in any one of claims 26 to 29, characterized in that the liquefied gas is chosen from hydrocarbons and dimethyl ether.

31. The use as claimed in claim 30, characterized in that the hydrocarbons are chosen from C₁₋₅ alkanes.

32. The use as claimed in any one of the preceding claims, characterized in that the liquefied gas is present in an amount ranging from 0 to 95% by weight, preferably between 0 and 60% by weight, with respect to the total weight of the propellant composed of the compressed and liquefied gases.

33. The use as claimed in any one of claims 26 to 32, characterized in that the weight polydispersity index (PI) is between 1.1 and 2.3.

34. The use as claimed in any one of claims 26 to 33, characterized in that the weight-average molecular weight of the compositionally gradient copolymer is between 5000 and 1 000 000 g/mol.

35. The use as claimed in any one of claims 26 to 34, characterized in that the number-average molecular weight of the compositionally gradient copolymer is between 5000 and 1 000 000 g/mol.

36. The use as claimed in any one of claims 26 to 35, characterized in that the compositionally gradient copolymer is such that, on the adsorption chromatography (LAC) curve representing the proportion of polymers as a function of the elution volume, the difference (V^{1/2} max−V^{1/2} min) is less than or equal to 3.5, preferably between 1 and 2.8, “V^{1/2} min” being the minimum value of the elution volume at mid-height of the curve and “V^{1/2} max” being the maximum value of the elution volume at mid-height of the curve.

37. The use as claimed in any one of claims 26 to 36, characterized in that at least one of the monomers of the compositionally gradient copolymer is a hydrophilic monomer.

38. The use as claimed in claim 37, characterized in that the hydrophilic monomer is chosen from:
amino(C₁-C₄ alkyl) (meth)acrylate derivatives;
di(C₁-C₈ alkyl)allylamines;
vinylamine;
vinylpyridines;
and their salts with inorganic acids or with organic acids, or their quaternized forms;
ethylenic carboxylic acids comprising from 3 to 20 carbon atoms, or their salts;
carboxylic anhydrides carrying a vinyl double bond and comprising from 4 to 30 carbon atoms;
ethylenic sulfonic or phosphonic acids, and their salts;
vinyl alcohol;
acrylamide or methacrylamide, N—(C₁-C₆ alkyl) (meth)-acrylamides, N,N-di(C₁-C₃ alkyl) (meth)acrylamides or N,N-di(C₁-C₄ alkyl)amino(C₁-C₆ alkyl) (meth)acrylamides;
hydroxy(C₂-C₄ alkyl) (meth)acrylates;
polyethylene glycol (5 to 100 ethylene oxide units) or glycol (meth)acrylates which are or are not substituted on their terminal functional group by C₁-C₄ alkyl, phosphate, phosphonate or sulfonate groups;
C₁-C₄ alkoxyalkyl (meth)acrylates;
polysaccharide (meth)acrylates;
optionally cyclic vinylamides;
vinyl ethers;
methacrylamidopropoxytrimethylammonium;
N,N-dimethyl-N-methacryloyloxyethyl-N-(3-sulfopropyl)-ammonium;
3-methacryloylethoxycarbonylpyridinium;
the compound of formula: 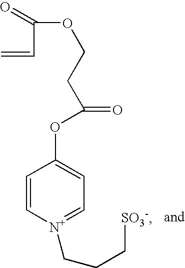
N-(3-sulfopropyl)-4-vinylpyridinium of formula:

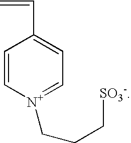

39. The use as claimed in claim 38, characterized in that the hydrophilic monomer is chosen from N,N-dimethylaminoethyl methacrylate (MADAME), acrylic acid, methacrylic acid, crotonic acid, styrenesulfonic acid, acrylamidopropanesulfonic acid, dimethylaminopropylmethacrylamide (DMAPMA), styrenesulfonate, hydroxyethyl acrylate, glyceryl acrylate, methoxyethyl (meth)acrylate, ethoxyethyl (meth)acrylate, methoxypolyethylene glycol (8 or 12 EO) (meth)acrylate, hydroxypolyethylene glycol (meth)acrylate, N-vinyl-pyrrolidone, N-vinylcaprolactam, acrylamide and N,N-dimethylacrylamide.

40. The use as claimed in any one of claims 26 to 39, characterized in that at least one of the monomers of the compositionally gradient copolymer is a hydrophobic monomer.

41. The use as claimed in claim 40, characterized in that the hydrophobic monomer is chosen from:
ethylenic hydrocarbons comprising from 2 to 30 carbon atoms;
acrylates of formula CH₂═CHCOOR₁, in which R₁ represents a saturated or unsaturated, linear, branched or cyclic, hydrocarbon group comprising from 1 to 30 carbon atoms in which one or more heteroatoms chosen from O, N, S and Si is/are optionally inserted, said hydrocarbon group additionally being optionally substituted by one or more substituents chosen from hydroxyl groups and halogen atoms,
or R₁ represents a —(R₁₀)ₓ—(OC₂H₄)ₙ—OR₁₁ group, with x=0 or 1, R₁₀=saturated or unsaturated, linear or branched, divalent hydrocarbon group comprising from 1 to 30 carbon atoms, n=5 to 100 and R₁₁=H or CH₃;
methacrylates of formula CH₂═C(CH₃)—COOR₂, in which R₂ represents a saturated or unsaturated, linear, branched or cyclic, hydrocarbon group comprising from 1 to 30 carbon atoms in which one or more heteroatoms chosen from O, N, S and Si is/are optionally inserted, said hydrocarbon group additionally being optionally substituted by one or more substituents chosen from hydroxyl groups and halogen atoms,
or R₂ represents —(R₁₀)ₓ—(OC₂H₄)ₙ—OR₁₁, with x=0 or 1, R₁₀=saturated or unsaturated, linear or branched, divalent hydrocarbon group comprising from 1 to 30 carbon atoms, n=5 to 100 and R₁₁=H or CH₃;
N—(C₈₋₃₀ alkyl) (meth)acrylamides;
vinyl esters of formula R₃—CO—O—CH═CH₂, where R₃ represents a linear or branched alkyl group having from 2 to 12 carbon atoms;
vinyl compounds of formula CH₂═CH—R₄, where R₄ is an —OC(O)—CH₃ group, a C₃ to C₈ cycloalkyl group, a C₆ to C₂₀ aryl group, a C₇ to C₃₀ aralkyl group (C₁ to C₄ alkyl group) or a 4- to 12-membered heterocyclic group comprising one or more heteroatoms chosen from O, N and S, said cycloalkyl, aryl, aralkyl and heterocyclic groups optionally being substituted by one or more substituents chosen from hydroxyl groups, halogen atoms and linear or branched alkyl groups having from 1 to 4 carbon atoms in which one or more heteroatoms chosen from O, N, S and P is/are optionally inserted and said alkyl groups additionally being optionally substituted by one or more substituents chosen from hydroxyl groups and halogen or Si atoms.

42. The use as claimed in claim 41, characterized in that the hydrophobic monomer is chosen from:
isoprene and butadiene;
methyl, ethyl, isobutyl, n-butyl, tert-butyl, ethylhexyl, furfuryl, isobornyl, tert-butylcyclohexyl or tert-butylbenzyl acrylates;
methyl, ethyl, n-butyl, isobutyl, hexyl or ethylhexyl methacrylates;
N—(C₆₋₁₂ alkyl)(meth)acrylamides;
vinyl esters of formula R₃—CO—O—CH═CH₂, where R₃ represents a linear or branched alkyl group having from 6 to 12 carbon atoms;
styrene;
vinyl acetate and vinylcyclohexane.

43. The use as claimed in any one of claims 26 to 42, characterized in that the compositionally gradient copolymer is present in the dissolved form or else in the form of an aqueous or organic dispersion.
